(19) United States
(12) Patent Application Publication
(10) Pub. No.: US 2014/0018673 A1
(43) Pub. Date: Jan. 16, 2014

(54) IONIZING RADIATION DETECTOR FOR USE WITH ENDOSCOPIC ULTRASOUND

(71) Applicants: Michael Keoni Manion, Cronulla (AU); George Charles Peppou, Hornsby Heights (AU)

(72) Inventors: Michael Keoni Manion, Cronulla (AU); George Charles Peppou, Hornsby Heights (AU)

(73) Assignee: Empire Technology Development LLC, Wilmington, DE (US)

(21) Appl. No.: 13/978,698

(22) PCT Filed: Jun. 18, 2012
(86) PCT No.: PCT/US12/42909

§ 371 Date: Jul. 8, 2013

(51) Int. Cl. A61B 8/00; A61B 8/08; A61B 6/00; G01T 1/20; A61B 8/12

(52) U.S. Cl. CPC A61B 8/4416; G01T 1/2006; A61B 8/12; A61B 6/425; G01T 1/2012; A61B 8/4483; A61B 6/481; A61B 8/5261
USPC 600/431; 600/436

(57) ABSTRACT

Embodiments provided herein relate to ionizing radiation detectors for use with endoscopic ultrasounds. In some embodiments, an apparatus is provided and includes an ultrasound transducer and an ionizing radiation detector, such as a scintillating detector. The ionizing radiation detector can comprise a scintillating material and a photodetector.

### FIELD

The present application generally relates to the field of endoscopic ultrasound.

### BACKGROUND

Endoscopic ultrasound is a medical procedure where endoscopy is combined with ultrasound to generate images of internal organs. Endoscopic ultrasound can be used to screen for cancers such as pancreatic cancer, esophageal cancer, and gastric cancer, as well as to observe other structures within a subject or tissue (for example, general tissue structures, gall stones, etc.).

### SUMMARY

In some embodiments, an apparatus for examination of tissue is provided. The apparatus includes an ultrasound transducer and an ionizing radiation detector, such as a scintillating detector, positioned proximally to the ultrasound transducer. The ionizing radiation detector can include a light detector and a scintillating material. In some embodiments, an apparatus for examination of tissue is provided. The apparatus includes an ultrasound transducer and an ionizing radiation detector; the ionizing radiation detector can include a light detector and a scintillating material. In some embodiments, an apparatus for examination of tissue is provided. The apparatus includes an ultrasound transducer and an ionizing radiation detector, such as a scintillating detector, in a three-dimensional shape such that it can detect ionizing radiation from different directions. The ionizing radiation detector can include a light detector and a scintillating material.

In some embodiments, a method for examination of tissue is provided. The method includes advancing an endoscope including an ultrasound transducer and a scintillating detector (or any ionizing radiation detector) to a tissue to be examined. The method can further include providing a subject with material that is a source of ionizing radiation.

In some embodiments, a method for generating a combined endoscopic ultrasound (EUS) and radiolabel image is provided. The method includes providing a probe including an endoscopic ultrasound head and a three-dimensional array of scintillating detectors (or other ionizing radiation detectors) positioned at a fixed location relative to the endoscopic ultrasound transducer. The method further includes obtaining an ultrasound image from the endoscopic ultrasound head and obtaining at least two-dimensional information from the three-dimensional array regarding a presence or absence of a radiolabel. The method further includes combining the ultrasound image with the at least two-dimensional information to provide a combined EUS and radiolabel image.

In some embodiments, a kit for endoscopic examination is provided. The kit includes an ultrasound transducer, a scintillating detector (or other ionizing radiation detector), and a radioactive material that is suitable for use in a subject and for inducing scintillation in the scintillating detector (or detection by the ionizing detector).

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a drawing depicting some embodiments of an apparatus for examination of tissue.

FIG. 2 is a drawing depicting some embodiments of a method for examination of tissue.

FIG. 3 is a drawing depicting some embodiments of a method for generating a combined endoscopic ultrasound (EUS) and radiolabel image.

FIGS. 4A-4D are drawings depicting hypothetical fields of view of different detectors.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Provided herein are embodiments that relate to the combination of ultrasound data, devices, and/or methods with ionizing radiation data, devices, and/or methods. By combining these aspects, as outlined herein, one can obtain devices, methods, and/or data, etc. that provide for greater insight into an area being examined.

In some embodiments, a device is provided that can serve as an ultrasound probe and a detector for ionizing radiation. The device can include an ultrasound transducer and an ionizing radiation detector.

In some embodiments, one or both of the ultrasound transducer and the detector are part of an endoscopic probe. The ultrasound transducer can be positioned near a distal end of the endoscopic probe. The detector can also be positioned near a distal end of the endoscopic probe.

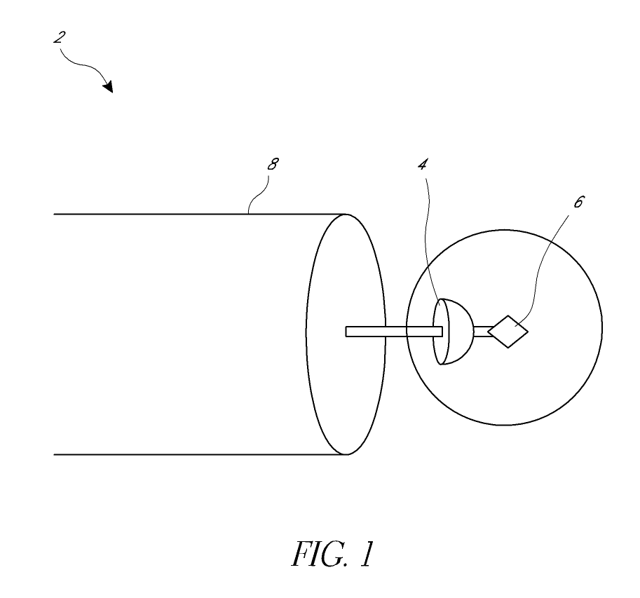

FIG. 1 depicts some embodiments of an apparatus 2 that includes an endoscopic probe 8 with a detector 4 and an optional acoustic coupling balloon 6 positioned at a distal end of the probe. In some embodiments, the detector and/or ultrasound transducer can be positioned elsewhere along the endoscopic probe.

In some embodiments, one or both of the ultrasound transducer and the ionizing radiation detector (which can be, for example, a scintillating detector) are not part of a same endoscopic probe. For example, in some embodiments, the ionizing radiation detector can be fed through the working channel of an endoscope towards an ultrasound transducer positioned at a distal end of the endoscope. In some embodiments, both the ionizing radiation detector and the ultrasound transducer can be fed through a working channel of an endoscope. In some embodiments, the ionizing radiation detector and the ultrasound transducer are not used in conjunction with an endoscope (which can be used, for example for FNA), but are each employed separately but during the same procedure.

In some embodiments, the ionizing radiation detector can be a scintillation detector, that can include a light detector and a scintillating material. The scintillating material can convert radioactive emissions into light. In some embodiments, the light detector is integrally formed with the scintillating material. In some embodiments, the light detector is spaced apart from the scintillating material.

In some embodiments, the scintillating material includes a polymer. The polymer can include a compound that includes an aromatic group. In some embodiments, the polymer that includes an aromatic residue, as part of the monomer, or, added as a co-solvent during polymerization, can scintillate, and therefore be used as a scintillating material. The emission of aromatic residues can occur in the UV range, for example, at approximately 300-350 nm. In some embodiments, the scintillating material includes at least one fluorophore. To decrease emission energy for imaging, primary fluors can be added to the polymer, allowing the light to be detected by light or photo detectors. The emissions can be attenuated and the wavelength of emission can be selected based on the selection of the fluors added. Any fluor can be used, depending upon the desired outcome and particular arrangement. In some embodiments, 2,5-Diphenyloxazole (PPO), terphenyl; 1,4-bis(5-phenyloxazol-2-yl) benzene (POPOP); 9,10-di-Phenylanthracene; 2-phenyl-5-(4-biphenylyl)-1,3,4-oxadiazole (PBD); or 2-(4′-tert-butylphenyl)-5-(4″-biphenylyl)-1,3,4-oxadiazole (B-PBD) can be used as the scintillating material. Combinations of these materials can also be used. As stated above, combinations can aid in producing light in a detectable range.

Other scintillating materials are also applicable. For example, the scintillating material can include one or more of the following polymers: a polystyrene, a polyvinyltoluene, a phenyl silicone, an epoxy with naphthalene, or an acrylic with naphthalene. In some embodiments, a second fluorescent scintillating material can be added to increase the efficiency of scintillation. In some embodiments, 2,5-diphenyloxazole (PPO), POPOP (1,4-bis(5-phenyloxazol-2-yl) benzene), 2-(4-tert-Butylphenyl)-5-(4-phenylphenyl)-1,3,4-oxadiazole (B-PBD) and/or 2-(4-Biphenylyl)-5-phenyl-1,3,4-oxadiazole (PBD) can be added as a second fluorescent scintillating material. In some embodiments, polyethylene naphthalate can be used as a scintillating material. Polyethylene naphthalate can function without added fluors. In some embodiments, epoxy and/or silicone based scintillating polymers can be used. Processing of the polymer can remain the same. For example, polysterenes are capable of injection molding. In some embodiments, scintillating materials can be used in liquid form or added to a polymer as a dopant to increase scintillating performance.

In some embodiments, the ionizing radiation detector (e.g., scintillating detector) includes an array. In some embodiments, a detector array can be read using a matrix to determine a change in impedance or resistance indicating the occurrence of detection (e.g., scintillation) events. In some embodiments, the detector includes at least one detector face. In some embodiments, the detector includes more than one detector (and can be an array), although they need not be arranged in any particular array configuration.

In some embodiments, the ionizing radiation detector includes a light detector or a photodetector (such as when a scintillation system is employed). Other detectors are also possible. For example, in some embodiments, the detector includes a miniature photodetector, a photo resistor, a CMOS sensor, and/or a CCD sensor. CMOS or CCD sensors can be used in combination with a bundle of fiber optics which can transmit light from a detector face to the sensors. This configuration can allow a higher resolution with a similar unit size.

In some embodiments, the detector is configured in a three-dimensional shape, so as to be able to provide additional information regarding the surroundings of the device, such as greater directionality in regard to the source of the ionizing radiation. In some embodiments, one or more detectors (or detector faces) can form the three-dimensional shape. For example, the three-dimensional shape can be hemispherical, approximately spherical, or multifaceted. Other shapes are also possible. For example, the three-dimensional shape can be ovular, rectangular, or triangular. In some embodiments, a multifaceted three-dimensionally shaped detector can approximate a three-dimensional shape, such as a hemisphere or sphere. In some embodiments, a single detector can be used for each facet or face of the detector. Multiple detectors can be used. In some embodiments, a detector face can measure incoming ionizing radiation from both direction, which side of the detector is being impacted can be resolved by measuring excitation and intensity, which can be measured as scintillation events per unit time. In some embodiments, the backside of the detector can be shielded, so as to provide greater and/or simpler directionality determinations.

In some embodiments, a distance between the ultrasound transducer and the scintillating detector is fixed. In some embodiments, the distance between the ultrasound transducer and the scintillating detector is variable. In some embodiments, the distance between the ultrasound transducer and the scintillating detector can from about 0 cm to about 1.5 cm. In some embodiments, the distance is from about 1 cm to about 2 cm. In some embodiments, the distance is from about 0 cm to about 5 cm. In some embodiments, the distance can be greater than 5 cm, however, as displacement between the ultrasound transducer and the scintillating detector increases the amount of information that may be extracted to augment the ultrasound image can be reduced. Other distances are also possible. Proximity between the ultrasound transducer and the scintillating detector can cause the field of view of the ultrasound transducer and the scintillating detector to be very similar. In some embodiments, known and/or fixed distances between the detector and the ultrasound transducer can be corrected for via in signal processing. Smaller, fixed, distances can allow for simpler corrections.

In some embodiments, the ultrasound transducer is part of a radial endoscopic ultrasound unit. Other ultrasound units are also possible. For example, in some embodiments, the ultrasound transducer is part of a linear endoscopic ultrasound unit.

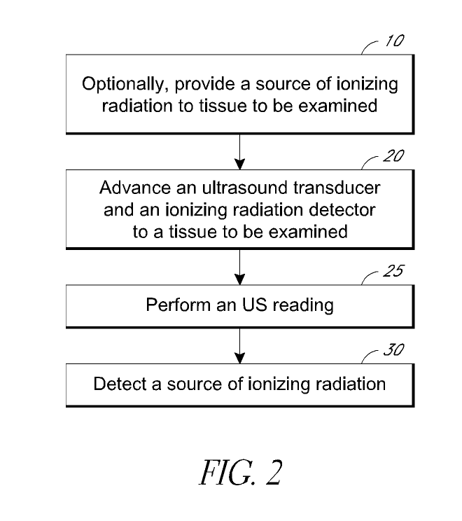

In some embodiments, a method of examination of tissue is provided. FIG. 2 depicts some embodiments of a method of examination. The method includes providing an ultrasound transducer and an ionizing radiation detector (which can be a scintillating detector), to a tissue to be examined. One can then detect a source of ionizing radiation in the subject 30 and also perform an ultrasound reading via the ultrasound transducer 25. In some embodiments, the method can also include providing a source of ionizing radiation to a tissue to be examined 10. In some embodiments, the method of examination of a tissue can utilize an apparatus such as the one described herein. As described in more detail below, in some embodiments, one can then combine the information from the ultrasound with the information from the ionizing radiation to provide an enhanced depiction of the surroundings of the endoscope.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

As noted above, in some embodiments, the method includes providing a subject with a material that is a source of ionizing radiation. In some embodiments, the material includes labeled glucose. For example, the source of ionizing radiation can include fludeoxyglucose (¹⁸F) (used in PET imaging) a positron emitter that can readily be detected through scintillation. In some embodiments, the labeled glucose can accumulate in a cancerous lesion at a rate much higher than surrounding tissue. Glucose molecules can also be labeled with ¹⁴C or ³H depending on the desired half-life. Other radiolabeled probes are also applicable. For example, FLT (3′-deoxy-3′-[18F]fluorothymidine), which marks proliferative tissue or radiolabeled monoclonal antibodies can also be used.

In some embodiments, the source of ionizing radiation is administered about 30 minutes or more before the endoscopic procedure and/or the detection of the ionizing radiation. In some embodiments, the source of ionizing radiation is administered about 30 to about 240 minutes before the endoscopic procedure and/or the detection of the ionizing radiation. In some embodiments, imaging, such as PET scanning can be performed after administration of the source of ionizing radiation and before imaging. In some embodiments, imaging, such as PET scanning can be performed about 30-40 minutes after administration of the source of ionizing radiation and before imaging via the ionizing radiation detector.

The amount of the source of ionizing radiation to be administered to a subject can depend on a number of factors, which can include the analyte, the label, and the detector used. For example, with labeled glucose, about 25 mL of the glucose analogue having about 5-10 mCi of radioactivity can be administered to a subject, however, more or less can be administered as desired.

In some embodiments, a patient can be instructed to fast before the procedure. Fasting can help to clear the GI tract prior to the procedure. Other GI tract clearing measures can also be taken. In some embodiments, a patient will be instructed to fast about 1-4 hours before the procedure. In some embodiments, a patient will be instructed to fast for about 4-6 hours before the procedure. In some embodiments, a patient will be instructed to fast for about 6-12 hours before the procedure.

In some embodiments, the material that is a source of ionizing radiation emits ionizing radiation that results in scintillation of the scintillating material of the scintillating detector. The scintillation of the scintillating material can produce light that hits the detector and is a wavelength detectable by the detector.

As noted above, in some embodiments, the method can further include collecting ultrasound data from the ultrasound transducer and collecting radiation data from the radiation detector (e.g., scintillating detector). In some embodiments, the method further includes comparing and/or combining the ultrasound data with the radiation data. The method can include providing an ultrasound image from the ultrasound data and providing a radiation determined image from the ionizing radiation detector data (which can be, for example, from a two dimensional and/or three-dimensional array). In some embodiments, comparing the two images can include a visual or digital comparison of the images. In some embodiments, the method includes providing an ultrasound image from the ultrasound data, providing a radiation based image from the radiation detector data, and combining the ultrasound image with the radiation based image to produce a combined image where the characteristics of each image are distinguishable from one another.

In some embodiments, the radiation image can be distinguishable from the ultrasound image in the combined image that is displayed to a user and/or manipulator of the probe. For example, the radiation based image can be highlighted a different color or otherwise identified. In some embodiments, the radiation image can be indistinguishable from the ultrasound image, so that the areas where they two images overlap will be intensified by having more of the same indicator (e.g., more or less shading and/or color).

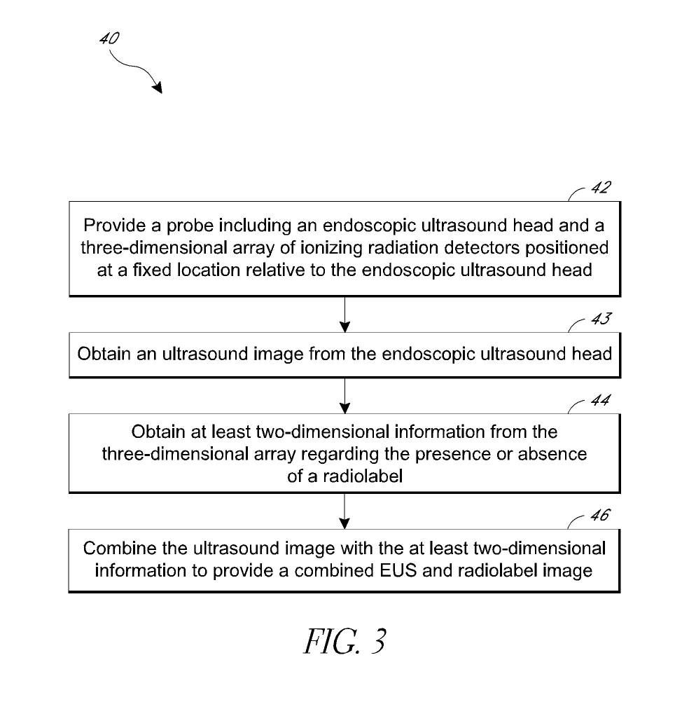

FIG. 3 depicts some embodiments of additional methods 40. The method includes providing a probe that includes an endoscopic ultrasound head and a three-dimensional array of detectors (or optionally a two dimensional array) positioned at a fixed location relative to the endoscopic ultrasound head 42. One can then position the probe within a subject to and obtain an ultrasound image from the endoscopic ultrasound head 43. The method also includes obtaining at least two-dimensional (if not three-dimensional) information from the array of detectors regarding a presence or absence of a radiolabel 44. The method further includes combining the ultrasound image with the at least two-dimensional information to provide a combined EUS and radiolabel image 46. In some embodiments, obtaining at least two-dimensional information from the three-dimensional array regarding a presence or absence of a radiolabel includes obtaining a three-dimensional image displaying the presence or absence of the radiolabel. In some embodiments, obtaining a three-dimensional image regarding the presence or absence of the radiolabel includes using a method of examination of tissue as described herein (e.g., the method of FIG. 2). In some embodiments, the method can further include displaying the combined image and/or recording the combined image to a computer readable medium.

In some embodiments, a kit for endoscopic examination is provided. The kit includes an ultrasound transducer, an ionizing radiation detector (such as a scintillating detector), and a radioactive material that is suitable for use in a subject and for inducing scintillation in the scintillating detector (or, more generally, for detection by an ionizing radiation detector). In some embodiments, the kit can include instructions regarding one or more of the methods provided herein. In some embodiments, the kit can include any of the other devices or aspects provided herein. In some embodiments, the kit can include an apparatus for administering the radioactive material, such as a syringe and/or cup. In some embodiments, the kit can include replaceable detectors and/or detector arrays. In some embodiments, the kit can include cleaning solutions for the detector and/or detector array. In some embodiments, the kit can include a balloon and/or sheath for the endoscope.

As noted above, in some embodiments, the ultrasound transducer and the detector can be provided at the distal end of an endoscopic probe. In some embodiments, the ultrasound transducer and the detector are separate from the probe and are configured to be inserted through the working channel of an endoscope. In some embodiments, one of the ultrasound transducer or the detector is provided at the distal end of an endoscopic probe and the other is configured to be inserted through the working channel of an endoscope. For example, the ultrasound transducer can be provided at the distal end of an endoscopic probe and the detector can be configured to be inserted through the working channel of an endoscope.

In some embodiments, the detector includes any detector that can detect a radio isotope. In some embodiments, the detector includes any detector that detects a radio isotope via a scintillating arrangement.

In some embodiments, the devices and/or methods can be especially useful in locating and/or identifying a cancerous lesion. In the course of a traditional EUS procedure locating a cancerous lesion can require the surgeon to search throughout the GI tract. Particularly with radial ultrasound, this can be difficult, due to the overall length of the GI tract. Fine needle aspiration (FNA) remains the ‘gold standard’ in endoscopic tissue characterization. However, due to size and time limitations the number of samples that can be taken in a single procedure can be limiting. This can become an issue when there are many polyps present within a cavity. In some embodiments, the present device and/or methods allow for a rapid, cost effective approach to determining which, if any, out of several lesions is cancerous, and/or can provide an increase in diagnostic effectiveness and efficiency.

Furthermore, even once a lesion has been found lesion edge detection and staging can remain a challenge due to lack of contrast of the tumor compared to surrounding tissue. EUS alone has a staging accuracy between 75% and 81%, with the lowest accuracy experienced when discriminating early stage tumors. While, EUS provides high sensitivity and specificity, the present inventors have appreciated that staging accuracy can be improved. Additionally, visualizing tumor cell accumulation in lymph nodes remains a key diagnostic factor that can be challenging to assess under previous technologies. Previously, proximal lymph node size has been used as a marker of metastasis; however, increased lymph node volume may be due to another unrelated condition. Determining if a lymph node contains a significant accumulation of tumor cells can have clear diagnostic advantages.

Thus, some of the disclosed embodiments, which combine radiography and ultrasound imaging as described herein, can increase patient comfort and convenience, and optionally, address one of more of the above noted challenges.

Furthermore, it is noted that, previously, patients have undergone a PET scan or CT scan and then a later endoscopy is conducted. To increase patient comfort the endoscopy can be performed without prior imaging and/or immediately following the PET scan. In some embodiments, the methods, apparatuses, and kits described herein can increase convenience for both patients and doctors as they can decrease procedure time.

In some embodiments, the methods, apparatuses, and kits described herein can increase the accuracy of imaging. Radiographic imaging, such as PET scanning and endoscopic ultrasound have demonstrated increased sensitivity for tumor detection (e.g., relative to CT scans). In some embodiments, combining the modalities can deliver a higher overall sensitivity. In some embodiments, the close proximity of the detector and the site of isotope accumulation can allow the detector to have a reduced sensitivity compared to external scanning arrays. In some embodiments, the materials used in some embodiments of the detection system are low cost PET scanner arrays, high cost scintillating crystals, and/or photomultiplier tubes. Such devices can be adequate in light of tissue attenuation and distance to a detector.

In some embodiments, the methods, apparatuses, and kits can enjoy simple data processing. Where each detector, (e.g., each face of a 3-dimensional scintillating detector) provides a discrete output, data processing can be simple to manage, as the input can be directly mapped to the corresponding direction that the surface faces, allowing for the various faces to provide for the directionality information that comes from the radioactive material.

While not required for all embodiments, in some embodiments, using a three-dimensional ionizing radiation detector can augment the entire field of view of the ultrasound transducer. A two-dimensional detector can require image focusing of detection events occurring on the ionizing radiation detector and in some situations may image a relatively narrow field in front of the detector. Matching the field of view (between the ultrasound transducer and the ionizing radiation detector) can allow the ionizing radiation detector to act as a contrast agent in any part of the ultrasound image. Due to the wide field of view of a three-dimensional array, such detectors can provide a signal to detect a lesion within a cavity in a superior manner. Radiation from directly above or below can provide clear directional data. In some situations, a two-dimensional detector may not be able to detect radiation emitted from above or below the detector, and thus, a three dimensional array arrangement of the detectors can provide further benefits.

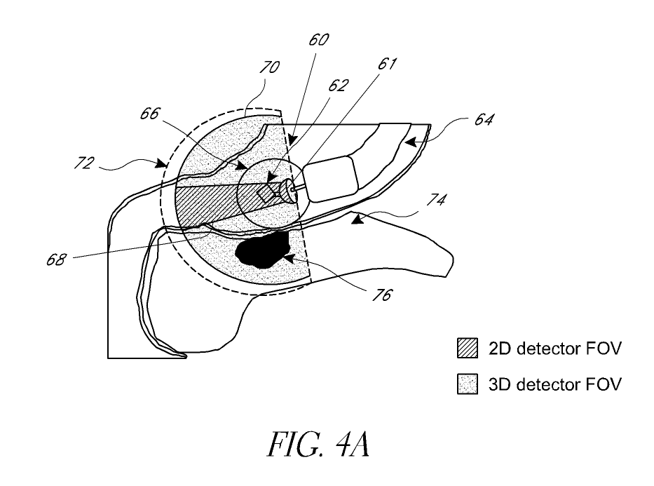

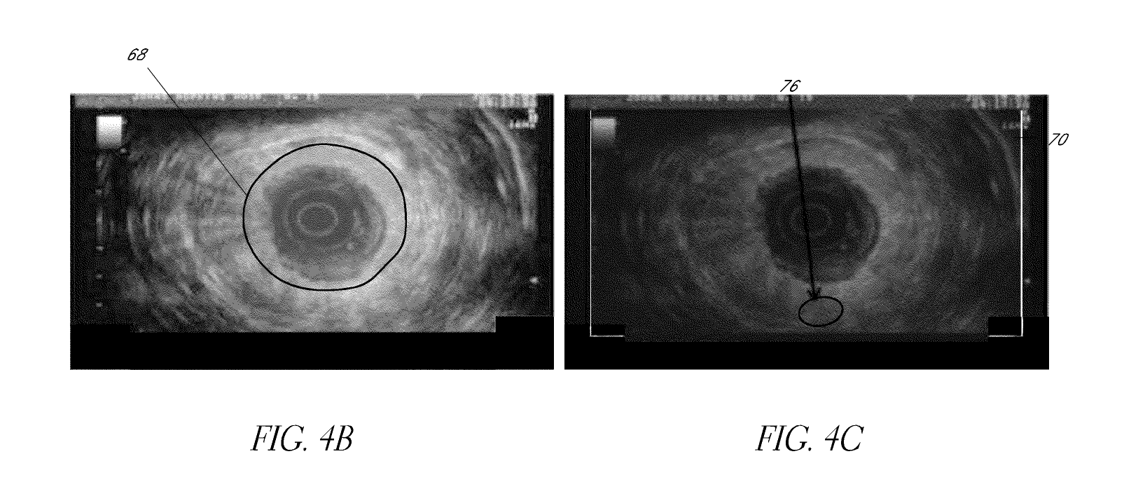

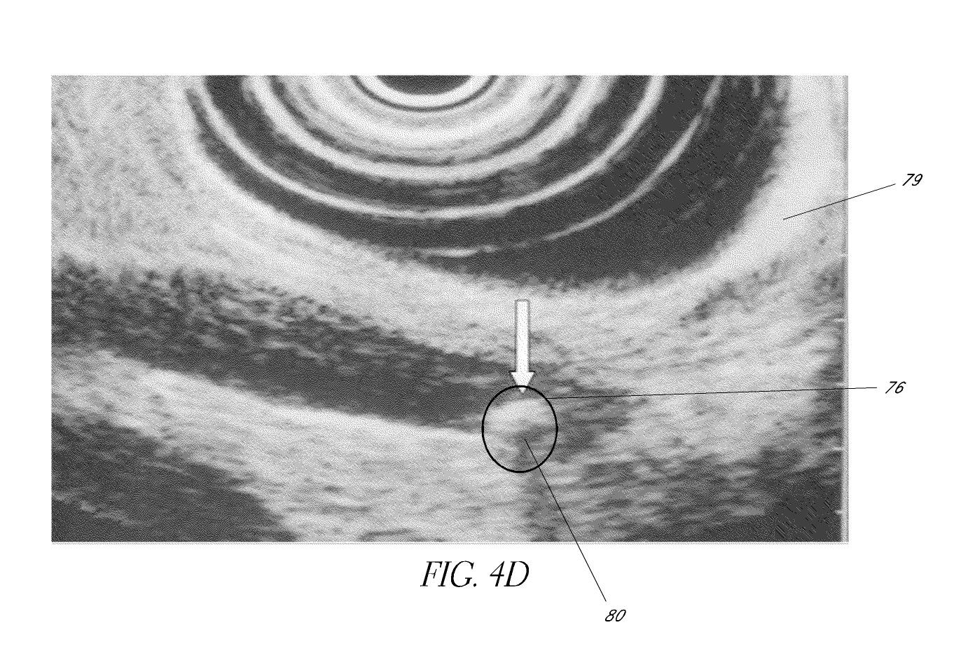

FIGS. 4A-4D are drawings depicting some embodiments of an apparatus 60 including an ultrasound transducer 62 and a three-dimensional detector 61 positioned on an endoscope 62. The field of view 70 of the three-dimensional detector 61 closely matches the field of view 72 of the ultrasound transducer 62. The field of view 68 of a two-dimensional detector is also shown. As depicted in FIG. 4A, the field of view 68 of a two-dimensional detector is narrower than the field of view 70 of the three-dimensional detector 61 and the field of view 72 of the ultrasound transducer 62. FIG. 4A shows the different FOVs of the disclosed 3-dimensional detector array compared to 2-dimensional arrays.

A cancerous lesion 76 is shown in FIG. 4A as containing a source of ionizing radiation within the normal tissue 74 near the apparatus 60. As is clear from FIG. 4A, such a lesion 76, would be readily detectable and locatable in the fields of view of both the three-dimensional array 70 and the ultrasound 72, but might otherwise fall outside of the field of view of the two dimensional array 68. However, in some embodiments, a two-dimensional array can also be sufficient.

FIG. 4B depicts a hypothetical field of view 60 of the two-dimensional detector. As shown, it is much smaller than the field of view 72 of the ultrasound transducer as the ultrasound image is much bigger than the field of view 60 of the two-dimensional detector. In contrast, FIG. 4C shows how the field of view 70 of the three-dimensional detector 61 closely matches the field of view 72 of the ultrasound transducer 62. In FIG. 4C, the three-dimensional detector has a field of view 72 wide enough to catch the cancerous legion 76. The comparison between the two figures demonstrates the predicted capability of the three-dimensional detector to visualize a wider field than a two-dimensional detector.

FIG. 4D depicts an image showing the combination of the ionizing radiation detector data 80 and the ultrasound data 79. The lesion 76 can be shown highlighted in a different color or emphasized by other approaches, such as shading, outlining, etc.

In an illustrative embodiment, any of the operations, processes, etc. described herein can be implemented as computer-readable instructions stored on a computer-readable medium. The computer-readable instructions can be executed by a processor of a mobile unit, a network element, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

### EXAMPLES

### Example 1

A patient is administered 20 mL of fludeoxyglucose (¹⁸F). The labeled glucose is preferentially taken up into a tumor in the subject's pancreas. About 2 hours later, an endoscope including an ultrasound transducer is advanced within the body of the patient to the pancreas for examination. A 3-dimensional scintillating detector is advanced through the working channel of the endoscope and is positioned near the ultrasound transducer.

The ultrasound transducer produces ultrasound waves and receives echoes. The scintillating detector registers both the intensity, in terms of scintillations per minute, and the direction, based on which detector faces are excited, of the radiotracer that has been administered to the body. The data received from the ultrasound transducer and detector is processed into an image format and combined to produce a combined image involving a background ultrasound image with the scintillating detector data highlighted in blue. The tumor in the subject's pancreas will contain more of the labeled glucose and therefore standout against the background level of radiation in the subject, allowing for the tumor to be identified via the scintillating detector data, which is mapped out onto the corresponding structures in the ultrasound. This allows for one to provide an enhanced image of the region of interest, with a special emphasis on the cancerous tissue.

### Example 2

A patient is administered a radio labeled antibody that specifically binds to a protein expressed in cancer cells along the digestive tract. The antibody binds to the cancerous cells and the unbound antibody is removed and/or broken down.

An endoscope including an ultrasound transducer and a two-dimensional scintillating detector positioned immediately behind the ultrasound transducer is provided. A user then advances the endoscope along the digestive track, taking repeated ultrasound and scintillating detector readings as the probe is advanced. Areas that indicate strong levels of ionization radiation from the radio labeled antibody are mapped out onto the corresponding ultrasound images that are taken at the same time. One can thereby scan a digestive track for cancerous areas and map those cancerous areas onto a corresponding ultrasound. One can further remove the cancerous tissue (e.g., ablation or surgery), and check via the combination of the ultrasound and the detector data to make certain that all of the cancerous tissue has been removed.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. An endoscopic ultrasound apparatus for examination of tissue, the apparatus comprising:
an ultrasound transducer; and
an ionizing radiation detector positioned proximally to the ultrasound transducer.

2. The apparatus of claim 1, wherein the ionizing radiation detector comprises a scintillating detector that comprises a light detector and a scintillating material.

3. The apparatus of claim 2, wherein the light detector is integrally formed with the scintillating material.

4. The apparatus of claim 2, wherein the light detector is spaced apart from the scintillating material.

5. The apparatus of claim 2, wherein the scintillating material comprises a polymer.

6. The apparatus of claim 5, wherein the polymer comprises a compound comprising an aromatic group.

7. The apparatus of claim 5, wherein the scintillating material comprises at least one fluorophore.

8. The apparatus of claim 5, wherein the polymer comprises one or more of a polystyrene, a polyvinyltoluene, a phenyl silicone, an epoxy with naphthalene, or an acrylic with naphthalene.

9. The apparatus of claim 1, wherein a distance between the ultrasound transducer and the ionizing radiation detector is 0 cm to about 1.5 cmfixed.

10. (canceled)

11. The apparatus of claim 1, wherein the ionizing radiation detector comprises a detector array.

12. (canceled)

13. The apparatus of claim 1, wherein the ionizing radiation detector comprises a three-dimensional shape.

14. The apparatus of claim 13, wherein the three-dimensional shape is approximately hemispherical, approximately spherical, or multifaceted.

15. The apparatus of claim 1, wherein the ultrasound transducer and the ionizing radiation detector are part of an endoscopic probe.

16. (canceled)

17. The apparatus of claim 1, wherein the ultrasound transducer is a radial endoscopic ultrasound.

18. A method of examination of tissue, the method comprising advancing an endoscope comprising an ultrasound transducer and an ionizing radiation detector to a tissue to be examined.

19. The method of claim 18, wherein the ionizing radiation detector is used to detect ionization radiation from three dimensions.

20. (canceled)

21. The method of claim 18, further comprising providing a subject with material that is a source of ionizing radiation.

22. The method of claim 21, wherein the material that is a source of ionizing radiation comprises at least one of: a labeled glucose or FLT (3′-deoxy-3′-[18F]fluorothymidine), which marks proliferative tissue.

23. (canceled)

24. The method of claim 21, wherein providing the subject with the material that is a source of ionizing radiation occurs about 0.5-4 hours after providing the subject with material that is a source of ionizing radiation.

25. (canceled)

26. The method of claim 18, wherein the ultrasound transducer and the ionizing radiation detector are separated by a distance of about 0 cm and about 5 cm.

27. (canceled)

28. The method of claim 18, wherein the ionizing radiation detector comprises a scintillating detector, and wherein the method further comprises providing a subject with material that is a source of ionizing radiation that emits ionizing radiation that results in scintillating a scintillating material in the scintillating detector, which produces light that hits a light detector of the scintillating detector.

29. The method of claim 18, further comprising collecting ultrasound data from the ultrasound transducer and collecting ionizing radiation detector data from the scintillating detector.

30. The method of claim 29, further comprising comparing the ultrasound data with the ionizing radiation detector data

31. (canceled)

32. (canceled)

33. The method of claim 30, further comprising:
providing an ultrasound image from the ultrasound data;
providing a scintillation image from the ionizing radiation detector data; and
combining the ultrasound image with the ionizing radiation image to produce a combined image.

34. The method of claim 33, wherein the ionizing radiation image is distinguishable from the ultrasound image in the combined image.

35. A method for generating a combined endoscopic ultrasound (EUS) and radiolabel image, the method comprising:
providing a probe comprising:
an endoscopic ultrasound head; and
a three-dimensional array of ionizing radiation detectors positioned at a fixed location relative to the endoscopic ultrasound head;
obtaining an ultrasound image from the endoscopic ultrasound head;
obtaining at least two-dimensional information from the three-dimensional array regarding a presence or absence of a radiolabel; and
combining the ultrasound image with the at least two-dimensional information to provide a combined EUS and radiolabel image.

36. The method of claim 35, wherein obtaining at least two-dimensional information comprises obtaining a three-dimensional image regarding the presence or absence of the radiolabel.

37. A kit for endoscopic examination, the kit comprising:
an ultrasound transducer;
an ionizing radiation detector; and
a radioactive material that is suitable for use in a subject and for inducing scintillation in ionizing radiation detector.
